(19) United States
(12) Patent Application Publication
(10) Pub. No.: US 2006/0210649 A1
(43) Pub. Date: Sep. 21, 2006

(54) Method and kit to cure chronic cough

(76) Inventor: Steven James Szarawarski, Spring Lake, NJ (US)

Correspondence Address: Steven James Szarawarski, 100 Newark Ave, Spring Lake, NJ, 07762, US

(21) Appl. No.: 11/084,935

(22) Filed: Mar. 21, 2005

(51) Int. Cl. A61K 33/06; H04K 3/00

(52) U.S. Cl. 424/682; 455/1

(57) ABSTRACT

The current invention is a method and kit to cure chronic cough, which is typically a dry cough. Chronic coughing is frequently caused by gastric acid which has been aerosolized by coughing, thereby causing hypersensitivity of the upper airway, leading to more coughing. This vicious cycle can torture the patient indefinitely. The cure consists of gargling with antacid solution when the urge to cough presents. The kit enables the patient to carry and administer the antacid solution on a timely basis. One version of the kit includes a cellular phone to ensure that the antacid gargle is readily available at all times. No phone call is made; the phone is simply something the patient is likely to have close by at all times and is big enough to carry a small bit of cargo within it's body. During a fit of extreme coughing, which is an extremely unpleasant experience, relief often comes within five seconds of administering the liquid antacid gargle. The total course of treatment for one person for one year was approximately equal to the cost of one (1) fancy cup of coffee (Not one coffee per day; just one.).

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The current invention is a cure for a non-productive chronic cough that will often follow a temporary cough. The current invention includes a convenient kit to facilitate the expeditious application of the pharmaceutical component. The method and kit to cure chronic cough is amazingly simple, effective, safe, and economical. The current invention replaces a long list of cough suppressant products, acid-production reducers, acid-neutralizing pills, medicated candies, throat lozenges, and other products and services which do not pinpoint the cause of the problem.

While the current invention will not eliminate 100% of chronic coughs, due to their multiple causes, the current invention is applicable to a large percentage of chronic coughs and should be considered in every case. The current state-of-the-art in chronic cough diagnosis and treatment is practiced with the aid of flowcharts consisting of dozens of nodes. The method and kit to cure chronic cough should be added near the top nodes of the flowcharts, above the more costly and more invasive modalities. Many patients will learn that they don't have to take medications indefinitely. For example, the strategy of taking proton pump inhibitors (forever) or H2 blockers (forever) gives the patient a lifetime of possible side effects, not to mention the cost and inconvenience. What the patient may have is a temporary upper airway hypersensitivity which can be cured in a few minutes.

The prior art includes many cough suppressant preparations containing the drug dextromethorphan, which is effective for certain productive coughs but famously ineffectual for non-productive chronic coughs. Other drugs, like proton pump inhibitors, are an example of gross over-medication which is entirely unnecessary due to the effectiveness of the current invention.

### BRIEF SUMMARY OF THE INVENTION

Temporary coughing is caused by common cold, allergy, influenza, dust, tobacco smoke, bronchitis, gastric reflux, postnasal drip, dry air, cold weather exercise, sneezing, numerous medications, and other factors. A chronic cough often follows a temporary cough and may persist indefinitely. Coughing leads to hypersensitivity of the upper airway, which leads to more coughing, which leads to more hypersensitivity, which leads to more coughing. Once hypersensitivity of the upper airway has been achieved, any dryness or gastric acid in the airway may cause fits of extreme coughing, including five or more coughs, of decreasing volume, per inhaled breath. Coughing puts pressure on the stomach which increases leakage of gastric acid into the esophagus. Further coughing creates an aerosol of gastric acid which lands on the upper airway and multiplies the hypersensitivity of the upper airway. All this results in, (you guessed it) more coughing! The cure for this vicious cycle is to gargle with an antacid solution when the urge to cough occurs, and to have the kit (containing the antacid solution) handy. Chewing antacid tablets is not sufficient, although it helps somewhat, in some cases. Extreme fits of coughing can often be eliminated within five seconds of the gargling treatment. Users of the method and kit to cure chronic cough should expect rapid dramatic improvement.

### BRIEF DESCRIPTION OF THE SEVERAL VIEWS OF THE DRAWING

Not Applicable

### DETAILED DESCRIPTION OF THE INVENTION

The pharmaceutical component, the liquid antacid, can be a common over-the-counter generic antacid formulation such as that found in liquid antacid preparations designed for treating excess stomach acid in humans, diluted with an equal volume of water. A common formulation contains water and 200 milligrams (dried gel) of aluminum hydroxide and 200 milligrams of magnesium hydroxide per 5 milliliters, and inert ingredients such as flavoring, before one-to-one dilution with water. A ready to use kit for curing chronic cough should be supplied with ready to use antacid solution, or water plus 100 milligrams of aluminum hydroxide and 100 milligrams of magnesium hydroxide per 5 milliliters. Additional ingredients for color, flavor, sweetness, etc. may be added as desired.

Neutralizing acidic environments can be an extremely imprecise task; there are an infinite number of combinations of neutralizing agents and an infinite number of combinations of liquid carriers. Therefore, the antacid formulation disclosed in the previous paragraph is an example of one convenient and highly effective formulation, but it is only one of many effective formulations.

A single gargling dosage consists of roughly three to ten milliliters of solution. A five second gargle with the head tilted well back followed by another few seconds gargling with that same solution should be sufficient. A low volume of air should be exhaled during the gargle; if a few drops of solution are lost in the airway, that's fine. The bulk of the solution remaining in the mouth may be swallowed or deposited in a sink, if available. Swallowing any remaining antacid solution will neutralize acid in the esophagus, which is helpful. Do not rinse nor clear the throat nor take a drink of anything afterward. The single dosage of antacid for gargling is less than the single dosage for heartburn or sour stomach; and it is much less if the excess gargling solution is not swallowed. Repeat as needed. It is helpful to resist the urge to cough while the antacid is working.

It is important to recognize what the upper airway hypersensitivity and cough reflex feels like. For example, it may feel like slight external pressure is being applied to the throat just below the adams apple, near the center of the collarbone. Or, a non-productive (dry) cough may be the only symptom. Or, there may be a constant urge to cough that is somewhat relieved by frequent small coughs. Typically, coughing does very little to reduce the urge to cough. Hot steam, as in a hot shower may help, but within ten minutes after showering, the cough urge is even stronger. The urge to cough may be very strong; it may feel like a wire brush is being pulled through the throat. Resting the voice for a few days does not help at all. The worst part of the day may occur minutes after going to sleep, when every breath becomes a battle not to cough. The patient may wake up with a big cough a few hours after going to sleep and have to again battle not to cough on each breath. The patient may have these same symptoms for a few months every year for decades in response to some seasonal stimuli. Typical cough suppressant products do not help at all. The patient should learn to neutralize the problem before developing his or her next upper airway hypersensitivity. Two treatments spaced ten minutes apart are sometimes helpful, but the patient's ability to understand what is going on is the most helpful, next to the information provided in this application.

The kit enables the person susceptible to chronic cough to carry a small quantity of antacid solution at all times. Antacid solution for heartburn is widely sold in screw-top 355 milliliter high density polyethylene bottles, which are simply not useful for someone on the go. The patient may go for many months without using their stash of antacid gargle, but since coughing leads to more coughing, it makes sense to be prepared. The kit may be refilled as needed from a large bottle stored at home.

A gelatin capsule; a wax capsule; a plastic capsule; a biteable capsule; or any small portable dosing mechanism could be used as the antacid container within the kit. The small portable doses could be integrated with a carrier for some other frequently carried object, such as a carrier for solid pieces, such as breath mints; paste, such as lip balm; suspensions, such as contact lenses; an electronic device; such as a wireless network communication appliance; or a media storage and media playing appliance, such as an audio music player.

As cellular phone components become smaller and smaller each year and human heads remain the same size, space becomes available within the cellular phone enclosure (the plastic body) to hold certain frequently carried items, thereby freeing the pants pockets from clutter. Small waterproof containers could easily be swapped in or swapped out of a cellular phone enclosure. Antacid capsules, breath mints, lip balm, keys, medications, contact lenses, makeup, payment devices, and other small items could be stored within the cargo hold of the cellular phone enclosure. Multiple doses of antacid solution to cure chronic cough would easily fit within the idle space of a cellular phone enclosure. Since months of time may elapse between dosages of antacid gargle, bundling the antacid gargle with the cellular phone is useful. The person prone to develop chronic cough may decide to carry the antacid capsules during seasonal allergy season only and use the cargo space for other items for the rest of the year. Many cellular phone users would find such a cargo space useful. Cargo is defined as something not part of the usual operation of the cellular phone. The phone's battery is not cargo; it's part of the phone. It is unlikely that the cellular phone handset makers will begin selling pharmaceutical products; therefore, the cellular phone would likely be sold with free samples of mints or candies in the cargo hold.

What I claim as my invention is:

1. A method for treating cough in a human, comprising the oral administration of a liquid antacid which is gargled by said human.

2. A pharmaceutical kit for treating cough in a human, comprising:
a. instructions indicating that liquid antacid gargle is for relieving cough;
b. a container for holding said liquid antacid gargle;
c. less than 101 milliliters of said liquid antacid gargle; and
d. an access means by which said human may gain rapid access to said liquid antacid gargle carried within said container.

3. A kit as in claim 2 which contains a secondary container for the storage of solid pieces, such as breath mints.

4. A kit as in claim 2 which contains a secondary container for the storage of paste, such as lip balm.

5. A kit as in claim 2 which contains a secondary container for the storage of wet products, such as contact lenses.

6. A kit as in claim 2 which contains an electronic device, such as a wireless network communication appliance.

7. A kit as in claim 2 which contains a media storage and media playing appliance, such as an audio music player.

8. A kit as in claim 2 which contains an attachment means, such as a belt clip.

9. A kit as in claim 2 which contains a key fob.

10. A kit for carrying less than fifty cubic centimeters of cargo, comprising:
a. a wireless network communication appliance with an outer body which opens to reveal a cargo storage area; and
b. an access means by which a human may gain rapid access to said cargo storage area.
